(19) United States
(12) Patent Application Publication
(10) Pub. No.: US 2007/0162045 A1
(43) Pub. Date: Jul. 12, 2007

(54) Curve linear and straight mandibular distractor with occlusion correction feature

(75) Inventor: Shaher A. Ahmad, Plano, TX (US)

Correspondence Address: BAKER BOTTS L.L.P., 2001 ROSS AVENUE, SUITE 600, DALLAS, TX, 75201-2980, US

(73) Assignee: OsteoMed L.P.

(21) Appl. No.: 11/317,483

(22) Filed: Dec. 22, 2005

(51) Int. Cl. A61B 17/60

(52) U.S. Cl. 606/105

(57) ABSTRACT

In accordance with one embodiment of the present invention, an osteodistraction device includes a flexible rod having a threaded portion. A distraction arm that includes a rod guide is coupled to the threaded portion of the flexible rod through a threaded hole near one end of the distraction arm. The rod guide of the distraction arm is operable to slide through a pivoting guide that is coupled to the distraction arm. An adjustment screw is coupled to the pivoting guide and is operable to rotate the distraction arm from a first to a second orientation. The flexible rod is operable to change the position of the rod guide relative to the pivoting guide.

### TECHNICAL FIELD OF THE INVENTION

The present invention relates generally to osteodistraction devices, and in particular to a curve linear and straight mandibular distractor with occlusion correction feature.

### BACKGROUND OF THE INVENTION

Bones sometimes develop at different rates, leaving some bones disproportionately shorter than other bones. Alternatively, injury may leave a bone shorter than its original length. Such a condition may lead to difficulties in a patient's movement. For instance, a patient with a shortened tibia may need special shoes for assistance in walking. A small jaw may cause difficulties in chewing or breathing. Moreover, deformations are often psychologically distressing to the patient, especially when the deformations occur in craniofacial bones.

One procedure for lengthening bones is referred to as osteodistraction. According to an osteodistraction procedure, an abnormally short bone is cut into two segments. The two segments are secured to a brace that permits the segments to be drawn apart. New bone then grows in the space between the separated bone segments, and eventually couples the two segments together into a lengthened bone. When the separated bone segments have been fully fused in this manner, the brace may be removed.

### SUMMARY OF THE INVENTION

In accordance with teachings of the present invention, the disadvantages and problems associated with osteodistraction of the mandible have been substantially reduced or eliminated. In particular, the system and method described herein provide for distraction of the mandible while also allowing for precise occlusion alignment.

In accordance with one embodiment of the present invention, an osteodistraction device includes a flexible rod having a threaded portion. A distraction arm that includes a rod guide is coupled to the threaded portion of the flexible rod through a threaded hole near one end of the distraction arm. The rod guide of the distraction arm is operable to slide through a pivoting guide that is coupled to the distraction arm. An adjustment screw is coupled to the pivoting guide and is operable to rotate the distraction arm from a first to a second orientation. The flexible rod is operable to change the position of the rod guide relative to the pivoting guide.

Certain embodiments of the present invention may also include rotating the distraction arm from the first orientation to the second orientation by rotating the pivoting guide and thereby rotating the distraction arm. The distraction arm may rotate around a central axis of the pivoting guide. Certain embodiments may also include the pivoting guide and adjustment screw being at least partially disposed within a housing, and the distraction arm passing through the housing. The flexible rod may be rotated and is thereby operable to change the position of the distraction arm relative to the housing.

In accordance with another embodiment of the present invention, an osteodistraction method may include coupling a distraction arm including a rod guide to a threaded portion of a flexible rod through a threaded hole near one end of the distraction arm. The rod guide may be operable to slide through a pivoting guide coupled to the distraction arm. An adjustment screw may also be coupled to the pivoting guide. The adjustment screw may be rotated, thereby rotating the distraction arm from a first orientation to a second orientation, and the flexible rod may be rotated, thereby changing the position of the distraction arm relative to the pivoting guide.

Technical advantages of certain embodiments of the present invention include the ability to adjust the angle between the distraction braces to allow the angles of the bones being distracted to be modified to correspond to the angles of bones which are not being distracted. Allowing these angle changes during distraction can avoid multiple surgeries and/or long and potentially painful orthodontic procedures.

Another technical advantage of certain embodiments of the present invention include a fully implantable and adjustable distraction device. The entire device may be disposed beneath the patient's skin and between the patient's gums and cheeks. This reduces visible scarring from the distraction by eliminating adjustment screws which pass through a patient's skin. These effects are beneficial to a patient's well being while not hampering a physician's ability to adjust the distractor.

Other technical advantages of the present invention will be readily apparent to one skilled in the art from the following figures, descriptions, and claims. Moreover, while specific advantages have been enumerated above, various embodiments mav include all, some, or none of the enumerated advantages.

### BRIEF DESCRIPTION OF THE DRAWINGS

For a more complete understanding of the present invention and its advantages, reference is now made to the following descriptions, taken in conjunction with the accompanying drawings, in which:

FIG. 1 illustrates one embodiment of a distractor as it may be installed within a patient;

FIGS. 2A and 2B are enlarged side views of two embodiments of a distractor in a partially expanded or distracted state;

FIG. 3 is an isometric view of one embodiment of a distractor with a portion of its housing broken away to illustrate the internal mechanics;

FIG. 4 illustrates an expanded view of one embodiment of a distractor;

FIG. 5 is an isometric view of an alternative embodiment of a distractor with a portion of its housing broken away to illustrate the internal mechanics; and

FIG. 6 is an isometric view of another embodiment of a distractor with a portion of its housing broken away to illustrate the internal mechanics.

### DETAILED DESCRIPTION OF THE INVENTION

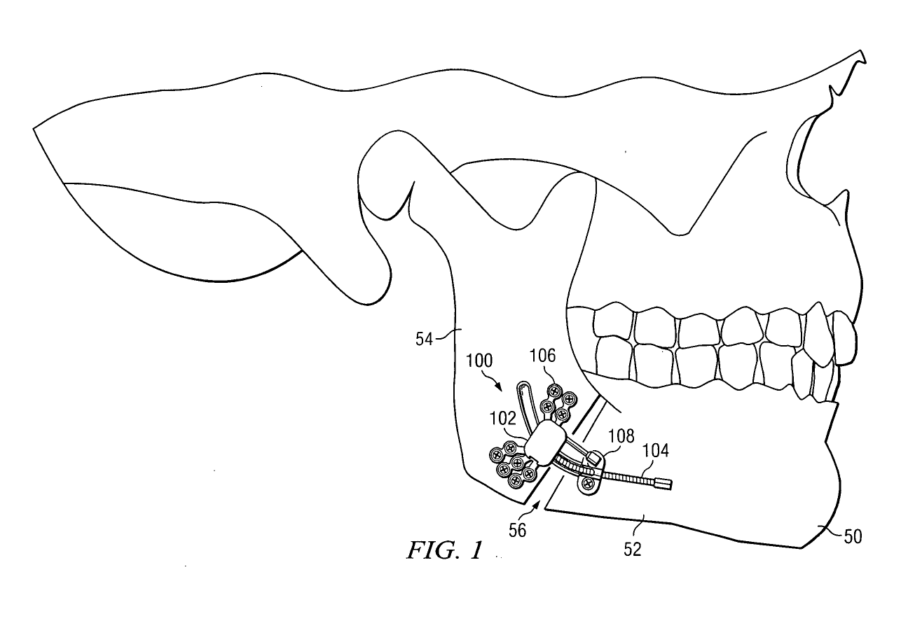

FIG. 1 illustrates one embodiment of distractor 100. Distraction devices, such as distractor 100, may be used to lengthen bones to correct congenital abnormalities and deformations following fractures or injuries. Osteodistraction is a procedure for lengthening bones by cutting a bone into two segments. The segments are drawn apart by a distraction device, such as distractor 100. New bone grows in between the separated bone segments, eventually coupling the two segments together, resulting in a lengthened bone. As the bone grows in between the separated segments, the segments may be drawn apart further until the desired bone length is achieved. Once the desired bone length is achieved, the device may remain on the bone until the newly grown bone is sufficiently strong, and then be removed.

In a particular embodiment, such as that illustrated by FIG. 1, distractor 100 could be a mandibular distraction device. Mandibular distraction devices may be used to lengthen the bones of the mandible, commonly known as the jaw. A cut is made through the bone of the mandible along a desired distraction line. A distraction device may be implanted such that it may impart a separating force on the bone segments of the mandible. The distraction device may be used to manipulate the bones to the desired position, and/or to a position that accommodates bone growth, and then be adjusted periodically as needed.

During a mandibular distraction procedure, the teeth of the lower dentition are also being distracted. A desirable result of the distraction procedure is a properly aligned occlusion. That is, that the teeth of the upper dentition properly align with the teeth of the lower dentition. A properly aligned occlusion is not inherent in a mandibular distraction procedure as patient conditions, bone structures, and amounts of distraction required are variable. Many patients may require a second procedure to correct their occlusion following distraction of their mandible. It is a goal of the present invention to provide mandibular distraction while also allowing significant adjustment of the angle between the portions of the mandible bone during distraction. Adjusting the angle between the portions of the mandible bone during distraction facilitates proper occlusion alignment and may reduce or eliminate the need for a separate occlusion alignment corrective procedure.

FIG. 1 illustrates distractor 100 as it may be installed in a human patient. While FIG. 1 illustrates one distractor 100 coupled to a patient's mandible bone, it should be understood that a patient may have a second distractor 100 coupled to their mandible bone on the opposite side (not shown). A cut 56 is made in mandible bone 50, separating lower portion 52 from upper portion 54. Distractor 100 includes a housing 102. Housing 102 is coupled to upper portion 54 by eyelets 106. An elongate arcuate distraction arm 104 passes through housing 102 and is mounted to lower portion 52 by plate 108. Eyelets 106 and plates 108 may be coupled to mandible bone 50 by bone screws or other appropriate securing system and/or method.

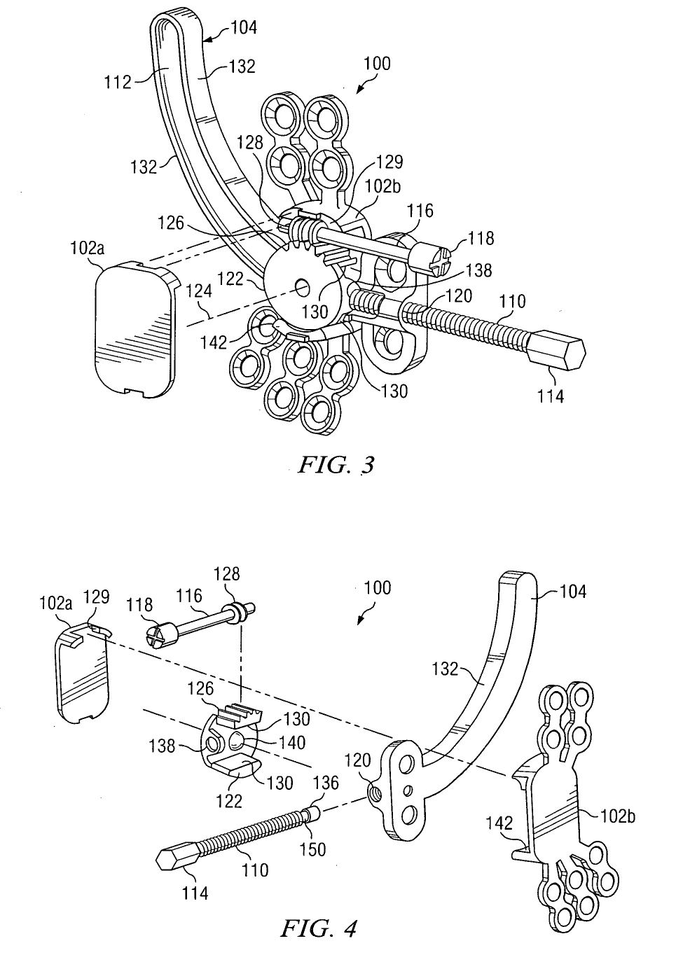

After installation of distractor 100, distraction arm 104 can be moved through housing 102 such that plates 108 move away from housing 102. Since lower portion 52 is coupled to distraction arm 104, and upper portion 54 is coupled to housing 102, the motion of distraction arm 104 through housing 102 may result in lower portion 52 moving away from upper portion 54. This movement increases the width of cut 56 and allows new bone to grow into cut 56. Ditractor 100 may also include occlusion correction capability. This occlusion correction capability may allow for adjustment of the angle of lower portion 52 relative to upper portion 54 such that the teeth of the upper dentition properly align with the teeth of the lower dentition. In this manner, mandible bone 50 can be adjusted and grown in multiple directions at the same time by distractor 100. This reduces or eliminates the need for multiple surgeries to correct deformations of mandible bone 50 which occur in more than one plane. The occlusion correction capability will be discussed in more detail with respect to FIGS. 3 and 4.

Components of distractor 100 and of other embodiments may comprise one or more of any suitable materials, such as titanium, titanium alloy, implantable stainless steel, nitnol and bioresorbable material.

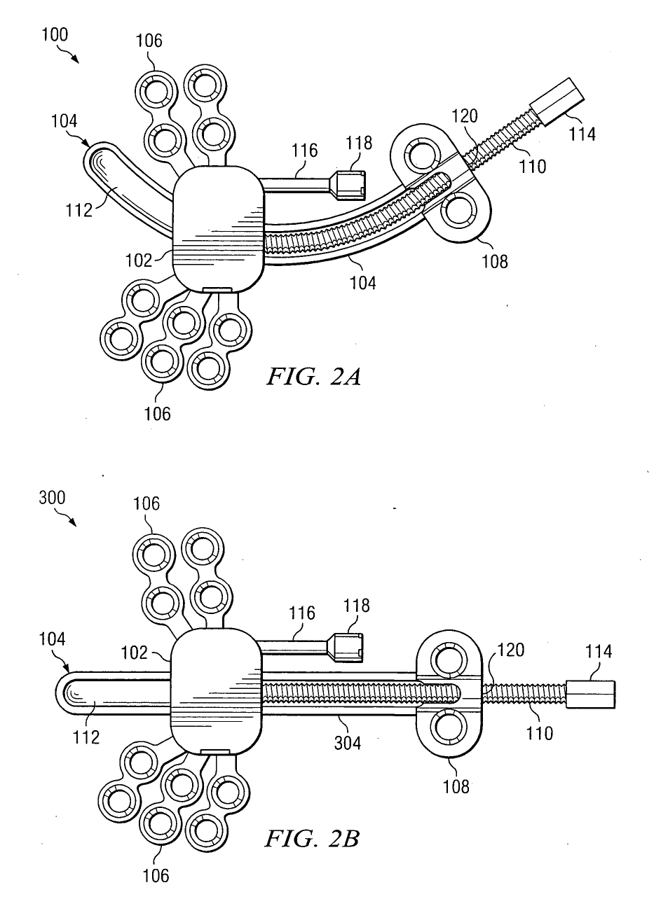

FIG. 2A illustrates a side view of distractor 100 in a partially expanded, or partially “distracted”, state. In the illustrated embodiment, distraction arm 104 passes through housing 102. In this embodiment, distraction arm 104 slides through housing 102, and housing 102 acts as a guide for distraction arm 104.

Distraction arm 104 is directed in its travels through housing 102 by flexible rod 110. Flexible rod 110 is an elongate rod containing threads through a central portion and interfaces on either end. In a particular embodiment, flexible rod 110 may be a nitinol rod. Constructing flexible rod 110 from nitinol allows flexible rod 110 to be deformed to a curvature corresponding to a curvature of distraction arm 104.

Flexible rod 110 passes through threaded hole 120 in the end of distraction arm 104. At one end, flexible rod 110 includes interface 114. Interface 114 may be used to rotate flexible rod 110 and thereby cause the external threads of flexible rod 110 to engage the internal threads of threaded hole 120. As this engagement occurs flexible rod 110 will be drawn into threaded hole 120 and thereby into distraction arm 104. Flexible rod 110 is prevented from passing through housing 102 by components of distractor 100 internal to housing 102 (discussed in more detail below). Therefore, as flexible rod 110 is threaded into distraction arm 104, threaded rod 110 pushes against the components internal to housing 102 and pulls distraction arm 104 through housing 102. Upper portion 54 and lower portion 52 are distracted as distraction arm 104 is drawn through housing 102. Therefore, distraction of upper portion 54 and lower portion 52 may be achieved by rotating interface 114 and causing flexible rod 110 to thread into distraction arm 104.

Distraction arm 104 includes rod guide 112. In the illustrated embodiment, rod guide 112 is a semi-circular indention in one side of distraction arm 104. In alternative embodiments, rod guide 112 may be a recessed channel with a square or rectangular shape, or may be an indention with practically any shape. Rod guide 112 serves to accept the portion of flexible rod 110 that passes through threaded hole 120.

Protruding from housing 102 is adjustment screw 116. Adjustment screw 116 may be used to adjust the occlusion alignment, as discussed in more detail below. Adjustment screw 116 includes interface 118. Interface 118 may be used to rotate adjustment screw 116.

In the illustrated embodiment plates 108 are shown as integral to distraction arm 104. Plates 108 may be cut or formed during a billeting or casting process. In alternative embodiments, plates 108 may be separate pieces welded to distraction arm 104, press fit onto distraction arm 104, or otherwise melded to distraction arm 104.

FIG. 2B illustrates an alternative embodiment of a distractor, labeled distractor 300. Distractor 300 operates in much the same way as distractor 100, discussed above. As can be seen comparing FIG. 2A to FIG. 2B, distraction arm 104 is curved or arcuate while distraction arm 304 is straight. While the operation of distractor 100 is similar to the operation of distractor 300, either may be better suited for a particular application such as a certain patients bone structure. Further alternative embodiments of the present invention may include a distraction arm taking any shape or curvature.

FIG. 3 illustrates an isometric view of distractor 100 in its retracted position with a portion of housing 102 broken away. The portion of housing 102 which has been broken away is labeled front side 102a. The remainder of housing 102 is labeled backside 102b. Backside 102b is illustrated as cradling various components of distractor 100, including the components of distractor 100 which enable distractor 100 to correct occlusional misalignments′. Distraction arm 104 passes through housing 102 and is prevented from separating from housing 102 by being sandwiched between a pivoting guide 122 and backside 102b. When front side of housing 102a is coupled to backside of housing 102b this further serves to secure distraction arm 104 within housing 102.

Flexible rod 110 passes through threaded hole 120 and into an alignment hole 138 in pivoting guide 122. Alignment hole 138 serves to keep flexible rod 110 properly seated within rod guide 112 as flexible rod 110 is threaded into distraction arm 104. In the illustrated embodiment, distraction arm 104 and rod guide 112 are curved. Therefore, as flexible rod 110 is fed into distraction arm 104, flexible rod 110 may be bent to correspond to the curvature of rod guide 112 so that flexible rod 110 will remain seated in rod guide 112. In alternative embodiments, distraction arm 104 and rod guide 112 may be straight, or curved to a greater or lesser degree. In many embodiments, it is desirable for distractor 100 to be compact. Recessing distraction arm 104 with rod guide 112 so that flexible rod 110 is seated within rod guide 112 makes distractor 100 more compact.

Pivoting guide 122 is held within backside 102b by rounded internal face 142 on the bottom side and by adjustment screw 116 on the top side. In this manner, pivoting guide 122 remains securely within housing 102 as distraction arm 104 travels through housing 102. Pivoting guide 122 also includes walls 130 (see FIG. 4) which interact with sides 132 of distraction arm 104. Sides 132 slide between walls 130 as distraction arm 104 passes through housing 102. Walls 130 hold distraction arm 104 in the proper alignment relative to housing 102 as distraction arm 104 passes through housing 102.

Pivoting guide 122 facilitates changing the angle of distraction arm 104 relative to housing 102 in order to properly align the teeth of the lower dentition with the teeth of the upper dentition. Adjusting the angle of distraction arm 104 relative to housing 102 is accomplished by rotating adjustment screw 116. Adjustment screw 116 and pivoting guide 122 act together to form a worm gear. The worm gear drive screw is adjustment screw 116, which includes raised threads 128. Raised threads 128 interact with teeth 127 on pivoting guide 122. The teeth 127 on pivoting guide 122 cause pivoting guide 122 to act as a gear. Raised threads 128 turn as adjustment screw 116 is turned. As raised threads 128 are turned, they engage teeth 126 and direct a rotational motion of pivoting guide 122.

Pivoting guide 122 may be rotated at any point during distraction. When pivotinq guide 122 is caused to rotate by the turning of adjustment screw 116, distraction arm 104 is also caused to rotate. Both pivoting guide 122 and distraction arm 104 rotate about central axis 124 of pivoting guide 122. This allows the point of rotation of distraction arm 104 to be adjusted during distraction. In this manner, adjustments may be made to the angle of distraction arm 104 relative to housing 102 during distraction to correct occlusional misalignments which may be appearing during the distraction or which may have existed prior to the distraction.

In the illustrated embodiment, adjustment screw 116 is held within housing 102 by cutout 129. Cutout 129 receives raised threads 128 and allows adjustment screw 116 to turn and properly engage teeth 126 while not allowing adjustment screw 116 to become separated from housing 102.

Pivoting guide 122 is held between rounded internal face 142 and raised threads 128 of adjustment screw 116. In this manner, pivoting guide 122 does not become separated from housing 102 as pivoting guide 122 is rotated.

Interface 118 of adjustment screw 116 is illustrated with an interface designed to interact with a Phillips Head screwdriver. Alternative embodiments of interface 118 could be designed to interact with other screwdriver types such as flathead, square, Allen, or torx. Interface 118 could also have a hex shape, or other geometric shape, to facilitate turning interface 118 with a wrench. Interface 114 of flexible rod 110 is illustrated with a hex head. Alternative embodiments of interface 114 could include a head having any geometrical shape or could include a screw head such as Phillips, flat, Allen, or torx.

FIG. 4 illustrates an exploded isometric view of distractor 100. This figure demonstrates how the components of distractor 100 are designed to fit with each other. Specifically, flexible rod 110 includes an end face 136 which is designed to pass through alignment hole 138 and contact protrusion 140. It is against protrusion 140 that flexible rod 110 will direct the force which will move distraction arm 104 through housing 102. Protrusion 140 is illustrated as having a raised half-spherical shape. This shape of protrusion 140 corresponds to the semi-circular depression of rod guide 112 (see FIGS. 2 and 3). The shape of protrusion 140 and the shape of rod guide 112 correspond to the shape and size of flexible rod 110. In this manner, flexible rod 110 may be threaded into distraction arm 104 and accepted by rod guide 112 as flexible rod 110 directs distraction arm 104 through pivoting guide 122. In the illustrated embodiment, the housing defining alignment hole 138 has also been rounded to correspond with the semi-circular shape of rod guide 112.

In some embodiments, alignment hole 138 may be semi-circular in shape, rather than a fully defined hole, and may have a sufficiently small radius that no portion of flexible rod 110 other than groove 150 may be fully seated within alignment hole 138. In this embodiment, flexible rod 110 would be securely coupled to pivoting guide 122 by the combination of groove 150 and alignment hole 138. In this manner, distraction arm 104 would be prevented from moving through housing 102 without rotating flexible rod 110.

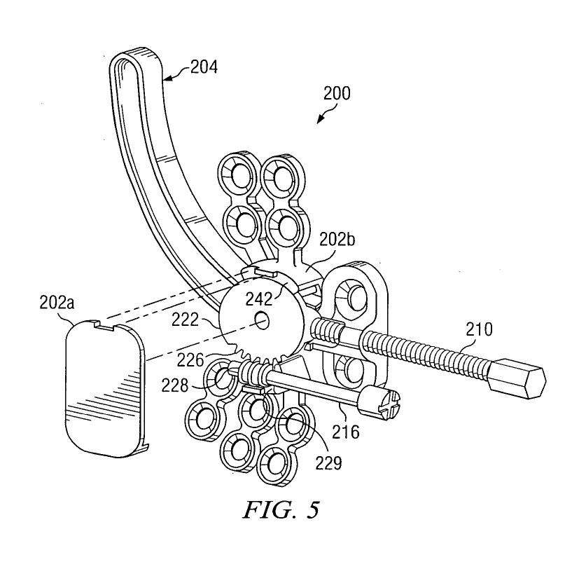

FIG. 5 illustrates an alternative embodiment of an osteodistraction device labeled distractor 200. FIG. 5 illustrates an isometric view of distractor 200 with front side 202a of housing 202 broken away. Distraction arm 204 passes through backside 202b. A pivoting guide 222 is seated within backside 202b. Pivoting guide 222 includes a plurality of teeth 226. Teeth 226 are engaged by raised threads 228 on adjustment screw 216. As adjustment screw 216 is rotated, raised threads 228 also rotate. As raised threads rotate they engage teeth 226 and thereby rotate pivoting guide 222. As pivoting guide 222 is rotated, it rotates distraction arm 204 about a central axis of pivoting guide 222.

Distractor 200 is similar to the previously described embodiment labeled distractor 100. As is apparent from the figures, several of the parts of distractor 200 that enable occlusion alignment have been moved from being above flexible rod 210 to being below flexible rod 210. Housing 202 has also been redesigned accordingly. Rounded interior face 242 is now on the top of backside 202b and cutout 229 has been moved to the bottom of backside 202b. It may be desirable in certain circumstances to have adjustment screw 216 under flexible rod 210. One example of such a circumstance might be particular patient limitation such as a small or unusually shaped mouth.

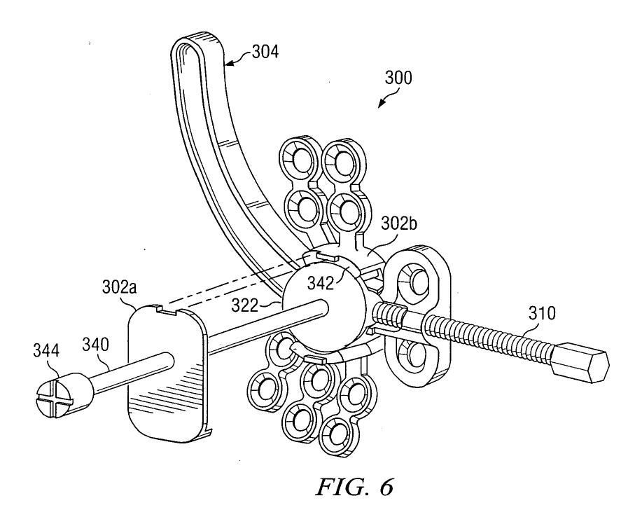

FIG. 6 illustrates an isometric view of a distractor 300, in accordance with a particular embodiment. Distractor 300 is similar to distractor 200 of FIG. 5, except that distractor 300 includes an adjustment arm 340 with a handle body 344 instead of adjustment screw 216 of FIG. 5. Handle body 344 is rotated to rotate pivoting guide 322. Similar to the embodiment of FIG. 5, this rotates distraction arm 304 about a central axis of pivoting guide 322. Other embodiments may include other configurations and mechanisms for rotating the distraction arm.

While various embodiments discussed herein include distractors and components that are utilized inside a patient's mouth, it should be understood that some embodiments may include one or more distractor components similar to those discussed herein that may be placed and utilized externally to the patient.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

What is claimed is:

1. An osteodistraction device, comprising:
a flexible rod having a threaded portion;
a distraction arm coupled to the threaded portion of the flexible rod through a threaded hole proximate one end of the distraction arm;
the distraction arm having a rod guide;
a pivoting guide coupled to the distraction arm;
the rod guide of the distraction arm being operable to slide through the pivoting guide;
an adjustment screw coupled to the pivoting guide;
the adjustment screw being operable to rotate the distraction arm from a first orientation to a second orientation; and
the flexible rod being operable to change the position of the rod guide relative to the pivoting guide.

2. The osteodistraction device of claim 1, wherein rotating the distraction arm from the first orientation to the second orientation includes rotating the pivoting guide and thereby rotating the distraction arm.

3. The osteodistraction device of claim 2, wherein the distraction arm rotates around a central axis of the pivoting guide.

4. The osteodistraction device of claim 1, wherein the pivoting guide prevents the distraction arm from rotating independently from the pivoting guide.

5. The osteodistraction device of claim 1, wherein:
the pivoting guide is at least partially disposed within a housing;
the distraction arm passes through the housing; and
the adjustment screw is at least partially disposed within the housing.

6. The osteodistraction device of claim 5, wherein the flexible rod may be rotated and is thereby operable to change the position of the distraction arm relative to the housing.

7. The osteodistraction device of claim 5, wherein the housing includes a plurality of eyelets operable to couple the housing to a portion of a mandible bone.

8. The osteodistraction device of claim 1, wherein the distraction arm includes a plurality of plates operable to couple the distraction arm to a portion of a mandible bone.

9. The osteodistraction device of claim 1, wherein:
the adjustment screw includes raised threads;
the pivoting guide includes a plurality of teeth; and
the raised threads are operable to engage the plurality of teeth and thereby rotate the pivoting guide relative to the adjustment screw.

10. The osteodistraction device of claim 1, wherein a length of the rod guide is curved.

11. An osteodistraction method, comprising:
coupling a distraction arm to a threaded portion of a flexible rod through a threaded hole proximate one end of the distraction arm;
the distraction arm having a rod guide;
coupling a pivoting guide to the distraction arm;
the rod guide being operable to slide through the pivoting guide;
coupling an adjustment screw to the pivoting guide;
rotating the adjustment screw and thereby rotating the distraction arm from a first orientation to a second orientation; and
rotating the flexible rod and thereby changing the position of the distraction arm relative to the pivoting guide.

12. The osteodistraction method of claim 11, wherein rotating the distraction arm from the first orientation to the second orientation includes rotating the pivoting guide and thereby rotating the distraction arm.

13. The osteodistraction method of claim 12, wherein the distraction arm rotates around a central axis of the pivoting guide.

14. The osteodistraction method of claim 11, wherein the pivoting guide prevents the distraction arm from rotating independently from the pivoting guide.

15. The osteodistraction method of claim 11, wherein:
the pivoting guide is at least partially disposed within a housing;
the distraction arm passes through the housing; and
the adjustment screw is at least partially disposed within the housing.

16. The osteodistraction method of claim 15, further comprising rotating the flexible rod and thereby changing the position of the distraction arm relative to the housing.

17. The osteodistraction method of claim 15, wherein the housing includes a plurality of eyelets operable to couple the housing to a portion of a mandible bone.

18. The osteodistraction method of claim 11, wherein the distraction arm includes a plurality of plates operable to couple the distraction arm to a portion of a mandible bone.

19. The osteodistraction method of claim 11, wherein:
the adjustment screw includes raised threads;
the pivoting guide includes a plurality of teeth; and
the raised threads are operable to engage the plurality of teeth and thereby rotate the pivoting guide relative to the adjustment screw.

20. The osteodistraction method of claim 11, wherein a length of the rod guide is curved.

21. An osteodistraction device, comprising:
a flexible rod having a threaded portion;
a distraction arm coupled to the threaded portion of the flexible rod through a threaded hole proximate one end of the distraction arm;
the distraction arm having an arcuate rod guide;
a pivoting guide coupled to the distraction arm;
the arcuate rod guide of the distraction arm being operable to slide through the pivoting guide;
an adjustment screw coupled to the pivoting guide;
the adjustment screw including raised threads;
the pivoting guide including a plurality of teeth;
the raised threads being operable to engage the plurality of teeth and thereby rotate the pivoting guide from a first orientation to a second orientation;
wherein rotating the pivoting guide also rotates the distraction arm;
wherein the pivoting guide prevents the distraction arm from rotating independently from the pivoting guide;
wherein the pivoting guide and the distraction arm each rotate around a central axis of the pivoting guide;
the flexible rod being operable to change the position of the distraction arm relative to the pivoting guide;
a housing at least partially surrounding the pivoting guide;
wherein the distraction arm passes through the housing;
wherein the adjustment screw is at least partially surrounded by the housing;
wherein the housing includes a plurality of eyelets operable to couple the housing to a portion of a mandible bone;
wherein the distraction arm includes a plurality of plates operable to couple the distraction arm to a portion of a mandible bone; and
the flexible rod being further operable to change the position of the distraction arm relative to the housing.
